(19) United States
(12) Patent Application Publication
(10) Pub. No.: US 2019/0083010 A1
(43) Pub. Date: Mar. 21, 2019

(54) BIO-SIGNAL DETECTING APPARATUS, AND APPARATUS AND METHOD FOR PROCESSING BIO-INFORMATION

(71) Applicant: SAMSUNG ELECTRONICS CO., LTD., Suwon-si (KR)

(72) Inventors: Seung Jun LEE, Seoul (KR); Eui Seok SHIN, Yongin-si (KR); June Young LEE, Seongnam-si (KR)

(73) Assignee: SAMSUNG ELECTRONICS CO., LTD., Suwon-si (KR)

(21) Appl. No.: 15/882,305

(22) Filed: Jan. 29, 2018

(30) Foreign Application Priority Data
Sep. 18, 2017 (KR) 10-2017-0119797

(51) Int. Cl. A61B 5/1455; A61B 5/145; A61B 5/00

(52) U.S. Cl. CPC A61B 5/1455; A61B 5/14546; A61B 2562/0242; A61B 5/7246; A61B 5/14532

(57) ABSTRACT

A bio-signal detecting apparatus, and an apparatus and method for processing bio-information are provided. The bio-signal detecting apparatus includes: a plurality of detection modules, each detection module of the plurality of detection modules including a detector disposed at a center thereof and a plurality of light sources disposed at a circumference centered at the detector, wherein the plurality of detection modules include a first detection module and a second detection module that is disposed adjacent to the first direction module, a circumference of the first detection module intersects with a circumference of the second detection module, and one or more light sources of the plurality of light sources are disposed at intersection points where the circumference of the first detection module intersects with the circumference of the second detection module.

### CROSS-REFERENCE TO RELATED APPLICATION

This application claims priority from Korean Patent Application No. 10-2017-0119797, filed on Sep. 18, 2017 in the Korean Intellectual Property Office, the disclosure of which is incorporated herein by reference in its entirety.

### BACKGROUND

### 1. Field

Apparatuses and methods consistent with exemplary embodiments relate to processing bio-information by detecting a bio-signal, and more particularly, to a bio-signal detecting apparatus, and an apparatus and method for processing bio-information.

### 2. Description of the Related Art

Research on the analysis and measurement of biomaterial by using a non-invasive method has attracted much attention, in which a bio-signal is measured by using a light detection device in a transmission mode or a reflection mode.

The transmission mode has an advantage in that an intensity of a measured bio-signal is large, but also has a drawback in that a measurement position should be limited in order to be applied to human skin. The reflection mode may be an ideal and convenient mode to be used in a system for measuring a bio-signal, but may have a problem in that an intensity of a measured signal is small.

In a general reflection mode system, a plurality of light sources and detectors are distributed randomly, in which a center distance between the light source and the detector is short, such that when a bio-signal of a subject is measured, an optical signal penetrating into only a thin layer of skin is measured. In order to improve this, a reflection mode system has been introduced, in which light sources and detectors are distributed with various center distances therebetween. However, the reflection mode system also has a problem in that detected bio-signals include bio-signals indicative of various depths, such that it is difficult to accurately detect a bio-signal desired to be measured.

### SUMMARY

Exemplary embodiments address at least the above problems and/or disadvantages and other disadvantages not described above. Also, the exemplary embodiments are not required to overcome the disadvantages described above, and may not overcome any of the problems described above.

According to an exemplary embodiment, there is provided a bio-signal detecting apparatus including: a plurality of detection modules, each of the plurality of detection modules including a detector disposed at a center thereof and a plurality of light sources disposed on a circumference centered at the detector, wherein the plurality of detection modules may include a first detection module and a second detection module that is disposed adjacent to the first direction module, a circumference of the first detection module may intersect with a circumference of the second detection module, and one or more of the plurality of light sources may be disposed at intersection points where the circumference of the first detection module intersects with the circumference of the second detection module.

The first detection module and the second detection module may share the one or more of the plurality of light sources disposed at the intersection points where the circumference of the first detection module with the second detection module; the plurality of detection modules may include a third detection module that is disposed adjacent to the first detection module; and a light source of the first detection module and a light source of the third detection module are disposed respectively at two intersection points where the circumference of the first detection module intersects with a circumference of the third detection module.

The one or more of the plurality of light sources shared by the first detection module and the second detection module may be disposed at the circumference of the third detection module.

A target distance between the detector and the plurality of light sources of each of the plurality of detection modules may be set based on a target depth from a surface of a subject to a location where a target biomaterial of the subject is present.

The target distance is 0.65 mm.

A number of the plurality of light sources of each detection module may be four and the plurality of light sources may be disposed at an equal interval on the circumference of each of the plurality of detection modules.

The plurality of light sources and the detector are physically separated by a light blocking part.

The apparatus may further include at least one of a parabolic mirror and a lens which focuses a light emitted from the plurality of light sources on a subject.

The apparatus may further include at least one of a parabolic mirror and a lens which focuses a light returning from a subject toward the detector.

According to an aspect of another exemplary embodiment, there is provided a bio-signal detecting apparatus including: a plurality of detection modules, each of the plurality of detection modules comprising a light source disposed at a center thereof, and a plurality of detectors disposed on a circumference centered at the light source, wherein the plurality of detection modules may include a first detection module and a second detection module that is disposed adjacent to the first detection module, a circumference of the first detection module may intersect with a circumference of the second detection module, and one or more of the plurality of detectors may be disposed at intersection points where the circumference of the first detection module intersects with the circumference of the second detection module.

The first detection module and the second detection module may share the one or more of the plurality of detectors disposed at the intersection points where the circumference of the first detection module with the second detection module; the plurality of detection modules may include a third detection module that is disposed adjacent to the first detection module; and a detector of the first detection module and a detector of the third detection module may be disposed respectively at two intersection points where the circumference of the first detection module intersects with a circumference of the third detection module.

The one or more of the plurality of detectors shared by the first detection module and the second detection module may be disposed at the circumference of the third detection module.

According to an aspect of another exemplary embodiment, there is provided a bio-information processing apparatus including: a bio-signal detector that comprises a plurality of detection modules, each of the plurality of detection modules comprising a plurality of light sources configured to emit light onto a subject, and a detector configured to detect light returning from the subject; and a processor configured to analyze a bio-signal obtained from the bio-signal detector and process bio-information, wherein the detector may be disposed at a center of a corresponding one of the plurality of detection modules, and the plurality of light sources may be disposed on a circumference centered on the detector, and the plurality of detection modules may include a first detection module and a second detection module that is disposed adjacent to the first detection module, wherein a circumference of the first detection module may intersect with a circumference of the second detection module, and one or more of the plurality of light sources may be disposed at intersection points where the circumference of the first detection module intersects with the circumference of the second detection module.

The processor may control the bio-signal detector and activate only light sources positioned at a plurality of reference distances from the detector, among the plurality of the light sources, to generate a reference signal for each of the plurality of reference distances.

The processor may correct the bio-signal based on the generated reference signal.

The processor may obtain the bio-information based on the corrected bio-signal by using a bio-information correlation model.

The bio-information may include information of at least one of moisture, protein, lipid, minerals, blood glucose, cholesterol, and neutral fats.

According to an aspect of another exemplary embodiment, there is provided a bio-information processing method, including: detecting a bio-signal by a plurality of detection modules, each of the plurality of module including a plurality of light sources configured to emit light on a subject, and a detector configured to detect the light returning from the subject; and analyzing the detected bio-signal to process bio-information, wherein the detector may be disposed at a center of a corresponding one of the plurality of detection modules, and the plurality of light sources may be disposed on a circumference centered on the detector, and the plurality of detection modules may include a first detection module and a second detection module that is disposed adjacent to the first detection module, and wherein a circumference of the first detection module may intersect with a circumference of the second detection module, and one or more of the plurality of light sources may be disposed at intersection points where the circumference of the first detection module intersects with the circumference of the second detection module.

The analyzing the bio-information may further include: activating only light sources positioned at a plurality of reference distances from the detector, among the plurality of the light sources, and generating a reference signal for each of the plurality of reference distances.

The analyzing the bio-information may further correcting the bio-signal based on the generated reference signal.

The analyzing the bio-information may further include obtaining the bio-information based on the corrected bio-signal by using a bio-information correlation model.

### BRIEF DESCRIPTION OF THE DRAWINGS

The above and/or other aspects will be more apparent by describing certain exemplary embodiments, with reference to the accompanying drawings, in which:

FIGS. 1A, 1B, and 1C illustrate a detection module of a bio-signal detecting apparatus according to an exemplary embodiment.

FIGS. 2A and 2B illustrate a detection module of a bio-signal detecting apparatus according to another exemplary embodiment.

FIG. 3 illustrates a bio-signal detecting apparatus according to an exemplary embodiment.

FIG. 4 illustrates a bio-information processing apparatus according to an exemplary embodiment.

FIG. 5 illustrates a bio-information processing method according to an exemplary embodiment.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail.

Process steps described herein may be performed differently from a specified order, unless a specified order is clearly stated in the context of the disclosure. That is, each step may be performed in a specified order, at substantially the same time, or in a reverse order.

Any references to singular may include plural unless expressly stated otherwise. In the present specification, it should be understood that the terms, such as ‘including’ or ‘having,’ etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

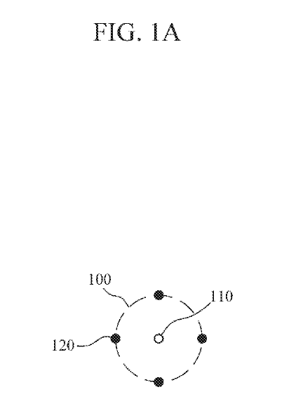

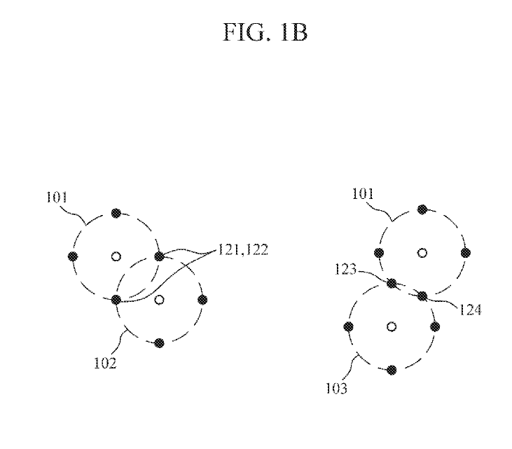

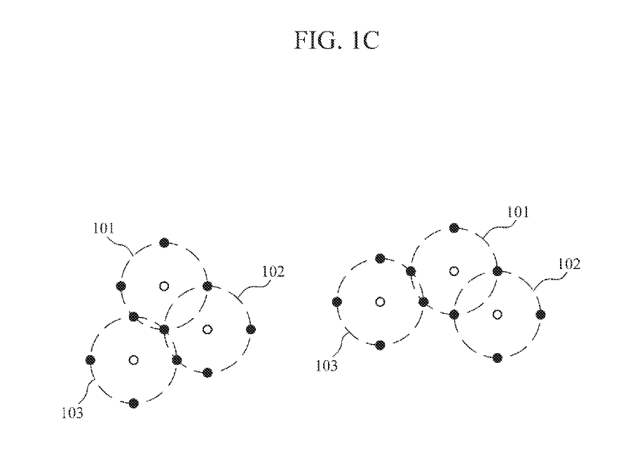

FIGS. 1A, 1B, and 1C illustrate a detection module of a bio-signal detecting apparatus according to an exemplary embodiment.

FIG. 1A is an exemplary diagram illustrating an arrangement structure of a detector 110 and light sources 120 of a detection module 100 of the bio-signal detecting apparatus. FIG. 1B is an exemplary diagram illustrating an arrangement structure between detection modules 101 and 102 or 101 and 103. FIG. 1C is an exemplary diagram illustrating an arrangement structure among detection modules 101, 102, and 103. The detection modules 101, 102, and 103 may be implemented as fiber optic probes.

Referring to FIG. 1A, the bio-signal detecting apparatus includes the detection module 100.

In the exemplary embodiment, the detection module 100 includes the detector 110 and the light source 120, and one or more of the light sources 120 may be disposed around the detector 110.

For example, the detection module 100 may include the detector 110 disposed at the center thereof, and a plurality of light sources 120 disposed on a circumference centered on the detector 110. For example, the plurality of light sources 120 may be four in number, which are disposed on the circumference at equal intervals. However, the arrangement of the light sources 120 is not limited thereto, and the plurality of light sources may be disposed at equal intervals on the circumference centered on the detector 110, in which only some light sources may be activated according to need.

Each detector 110 may detect light reflected or scattered from a subject, and may measure the intensity of the detected light. In the embodiment, the detector 110 may include a photo diode, photo transistor (PTr), a charge-coupled device (CCD), or the like.

Further, the light source 120 may emit light on a subject. For example, the light source 120 may emit light of a predetermined wavelength (e.g., near infrared (NIR)) on the subject. However, the wavelength of light emitted by the light source 120 may vary depending on the purpose of measurement or types of components to be measured. Further, the light source 120 is not necessarily required to be configured as a single light emitting body, but may be configured as a group of a plurality of light emitting bodies. In the case where the light source 120 is configured as a group of a plurality of light emitting bodies, the light emitting bodies may emit light of different wavelengths to serve the purpose of measurement, or may emit light of the same wavelength. In the exemplary embodiment, the light source 120 may include a light emitting diode (LED), a laser diode, or the like, but this is only exemplary, and the light source 120 is not limited thereto.

The distance between the detector 110 and the light source 120 of the detection module 100 may be set based on a depth from the surface of a subject (e.g., skin surface) to a location of a biomaterial (e.g., blood glucose) to be measured.

Here, the biomaterial to be measured may be referred to as a target, the depth from the surface to the target may be referred to as a target depth, and the distance between the detector 110 and the light source 120 may be referred to as a target distance.

Further, the biomaterial may be skin components, such as moisture, protein, lipid, or various minerals, and may include at least one of blood glucose, cholesterol, and neutral fats as blood components.

For example, the intensity of an optical signal detected by the detector 110 may be increased as the intensity of light emitted on a subject is higher, and the distance between the detector 110 and the light source 120 is shorter. In this case, the distance between the detector 110 and the light source 120, namely the target distance, has a correlation with the target depth. For example, in the case where the bio-signal is measured by using the reflection mode, the bio-signal may be measured by detecting light, which is emitted again after being transmitted to a certain depth point of the subject. In this case, if the target is located deep from the surface of skin of the subject, an optical wavelength may be required to reach deep inside the subject, such that the target distance is determined to be relatively long in order to detect an optical signal which is reflected or scattered after being transmitted deep into the subject.

In the case where the target is at a shallow depth from the surface of a subject, an optical wavelength may only need to reach a point near the surface of the subject, such that the target distance is determined to be relatively short in order to detect an optical signal which is reflected or scattered after being transmitted to a point near the surface of the subject. As described above, there is a correlation between the target distance and the target depth, and the target distance may be determined according to the types of bio-information of a subject to be measured and the target depth.

For example, in order to detect a bio-signal of a dermal layer, the target depth may be about 1.0 mm to 2.0 mm, in which case the target distance may be determined to be about 0.65 mm. By comparison, if the target depth is deeper than the dermal layer (e.g., 2.0 mm or deeper depths), the target distance may be longer than about 0.65 mm; and if the target depth is shallower than the target depth (e.g., 1.0 mm or shallower depths), the target distance may be shorter than about 0.65 mm.

As described above, the bio-signal detecting apparatus determines the target depth according to the types of biomaterial to be measured, and determines the target distance based on the determined target depth, thereby accurately detecting only the signal of biomaterial to be measured.

Referring to FIGS. 1A and 1B, the bio-signal detecting apparatus includes a plurality of detection modules 101, 102, and 103. Here, the plurality of detection modules 101, 102, and 103 may be detection modules, each having the same arrangement of the detector 110 and the light source 120.

Hereinafter, for convenience of explanation, the detection module 101 will be referred to as a first detection module 101, the detection module 102 as a second detection module 102, and the detection module 103 as a third detection module 103. As will be described below in detail, the plurality of detection modules may be arranged repeatedly and regularly, such that the names to refer to the plurality of detection modules are not limited thereto.

In the bio-signal detecting apparatus, the first detection module 101 and other detection modules 102 and 103 adjacent thereto are arranged so that the circumferences thereof intersect each other, and the light sources may be disposed at each intersection point.

In the exemplary embodiment, the first detection module 101 and the adjacent second detection module 102 of the bio-signal detecting apparatus may share the light sources 121 and 122 which are disposed at two points where the circumferences of the first detection module 101 and the adjacent second detection module 102 intersect each other.

For example, the first detection module 101 and the second detection module 102 are detection modules having the same arrangement of the detector 110 and the light sources 120, in which the detector 110 is disposed at the center thereof, and the light sources 120 are disposed at equal intervals on the circumference centered on the detector 110. In this case, the first detection module 101 and the second detection module 102 are adjacent to each other, such that the circumferences thereof centered on the detector may be arranged to intersect at least at two points, and the light sources 121 and 122 disposed at the intersection points may be shared by the first detection module 101 and the second detection module 102.

Further, the first detection module 101 and the third detection module 103 adjacent thereto may be arranged so that the circumferences thereof may intersect each other. For example, the first detection module 101 and the adjacent third detection module 103 may be arranged so that each one light source of the first detection module 101 and the third detection module 103 may be disposed respectively at two intersection points of the circumferences thereof. For example, when the circumferences of the first detection module 101 and the adjacent third detection module 103 intersect or overlap each other, one light source 123 of the third detection module 103 may be disposed on the circumference of the first detection module 101, and one light source 124 of the first detection module 101 may be disposed on the circumference of the third detection module 103.

Referring to FIGS. 1A, 1B, and 1C, the plurality of detection modules 101, 102, and 103 of the bio-signal detecting apparatus may be arranged with their circumferences intersecting each other.

In another exemplary embodiment, the first detection module 101 and the second detection module 102 are adjacent to each other, so that the circumferences thereof centered on the detector may intersect at least at two points; the light sources disposed at the intersection points may be shared by the first detection module 101 and the second detection module 102; and each one light source of the first detection module 101 and the adjacent third detection module 103 may be disposed respectively at two intersection points of the circumferences thereof.

For example, when the circumferences of the first detection module 101 and the adjacent third detection module 103 intersect each other, one light source of the third detection module 103 may be disposed on the circumference of the first detection module 101, and one light source 124 of the first detection module 101 may be disposed on the circumference of the third detection module 103. In this case, at least one of the shared light sources 121 and 122 of the first detection module 101 and the second detection module 102 may be disposed on the circumference of the third detection module 103.

For example, the circumference of the third detection module 103 is arranged so as to intersect with each circumference of the first detection module 101 and the second detection module 102 at least at two points, in which at least one of the shared light sources 121 and 122 of the first detection module 101 and the second detection module 102 may be disposed on the circumference of the third detection module 103; one of the light sources of the third detection module 103 may be disposed at a point where the circumferences of the first detection module 101 and the third detection module 103 intersect each other; and the other one of the light sources of the third detection module 103 may be disposed at a point where the circumferences of the second detection module 102 and the third detection module 103 intersect each other.

In yet another exemplary embodiment, in the detection module of the bio-signal detecting apparatus, the positions of the light source and the detector may be switched with each other. For example, the bio-signal detecting apparatus may include a plurality of detection modules, each including the light source disposed at the center thereof, and a plurality of detectors disposed on a circumference centered on the light source.

In this case, the first detection module and another detection module that is adjacent thereto are arranged so that the circumferences thereof intersect each other, and the detectors may be disposed at each intersection point. Further, the first detection module and the adjacent second detection module may share the detectors disposed at two intersection points of the circumferences thereof; and each one detector of the first detection module and the adjacent third detection module may be disposed respectively at two points of the circumferences thereof.

Further, at least one of the detectors shared by the first detection module and the second detection module may be disposed on the circumference of the third detection module.

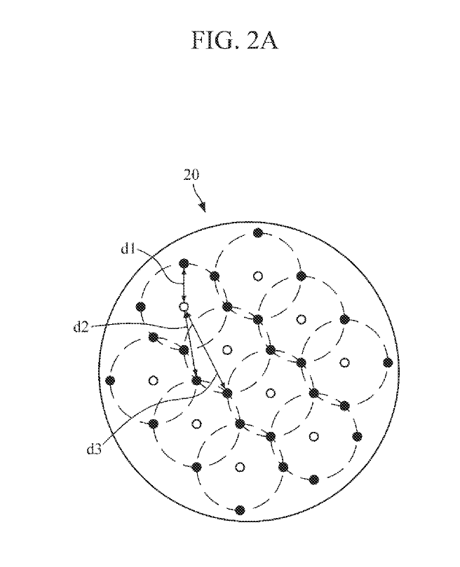

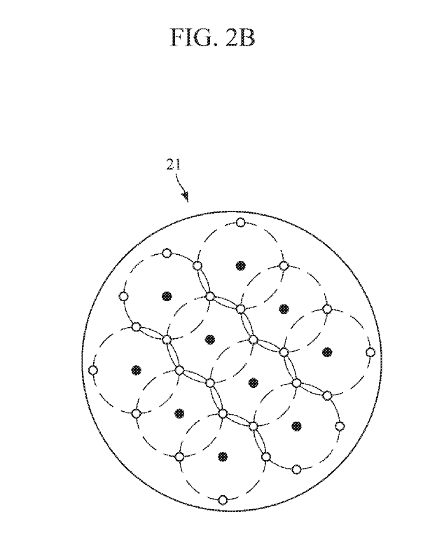

FIGS. 2A and 2B are exemplary diagrams illustrating another example of a detection module of a bio-signal detecting apparatus.

Referring to FIGS. 1A and 2A, the detection modules 100 of a bio-signal detecting apparatus 20 may be arranged regularly and repeatedly within a predetermined area.

In the exemplary embodiment, in the case where the bio-signal detecting apparatus 20 includes a probe which interfaces with a subject to measure a bio-signal, the detection modules 100 may be arranged regularly and repeatedly within a diameter of the probe, and the number of the detection modules 100 to be arranged regularly and repeatedly may vary depending on the diameter of the probe. Here, the repetitive arrangement of the plurality of detection modules of FIG. 2A is a repetitive arrangement structure of adjacent detection modules as described above with reference to FIGS. 1B and 1C, such that overlapping description thereof will be omitted.

A plurality of light sources included in the repeatedly arranged detection modules of the bio-signal detecting apparatus 20 may be disposed at various distances d1, d2, d3, . . . , and dn from the detector 110 of each detection module 100.

For example, the detector 110 of any one detection module 100 may detect light emitted from a light source at the target distance d1, and may detect light emitted from light sources, positioned at distances other than the target distance d1, of other detection modules. In this case, the distances d2, d3, . . . , and dn between the detector and light sources, other than the target distance d1, may be longer than the target distance d1; and the number of the light sources positioned at equal distances from the detector of any one detection module may vary in accordance with the number of the plurality of detection modules of the bio-signal detecting apparatus 20.

Further, the detector 110 included in any one detection module 100 of the bio-signal detecting apparatus 20 may detect light, which is reflected and/or scattered after being emitted from the light sources of other detection modules that are positioned at various distances d2, d3, . . . , and dn, other than the light source 120 positioned at the target distance d1. In this case, an optical signal detected by the detector 100 may be in inverse proportion to a distance between the detector and the light source, and may be in proportion to the intensity of light emitted from the light source, and the number of light sources.

For example, referring to FIG. 2A, in the case where the bio-signal detecting apparatus 20 detects a bio-signal through a probe having a diameter of 4.4 mm, the bio-signal detecting apparatus 20 includes ten detection modules 100 with the target distance d1 of 0.65 mm, and each of the detection modules 100 may be arranged regularly and repetitively with adjacent detection modules.

For example, the number of the light sources positioned at the target distance (d1=0.65 mm) from the detector of each of the plurality of detection modules may be 54; the number of light sources positioned at the second nearest distance (d2=1.1 mm), after the target distance (d1=0.65 mm), from the detector of each of the plurality of detection modules may be 24; and the number of the light sources positioned at the third nearest distance (d3=1.4 mm), after the second nearest distance, from the detector of each of the plurality of detection modules may be 24.

In this case, a relationship of an illustrative distance between the target distance (d1=0.65) and the distances (d2=1.1 and d3=1.4) to the light sources, which are longer than the target distance, may be calculated by using

$${d}_{r 12} = \frac{dr 2}{dr 1} = 1.7 and {d}_{r 13} = \frac{dr 3}{dr 1} = 2.2 .$$

A relationship between the number of light sources and distances from the detectors may be calculated by using

$${d}_{l 12} = \frac{d 2}{d 1} = 0.44 and {d}_{l 13} = \frac{dl 3}{dl 1} = 0.44 .$$

As described above, the bio-signal detecting apparatus 20 has the largest number of light sources at the target distance d1; the number of light sources positioned at other distances d2, d3, . . . and dn is reduced to half or less; and the light sources positioned at other distances d2, d3, . . . and dn are positioned at distances about twice the target distance d1.

As a result, according to the arrangement structure of the detection modules of the bio-signal detecting apparatus 20, the intensity of an optical signal detected from the light sources positioned at the distances d2, d3, . . . and dn, other than the target distance d1, is exponentially reduced, such that only the bio-signal to be measured may be measured accurately. The target distance (d1=0.65), which is provided for convenience of explanation, is not limited thereto, and may be determined based on a target depth which is a bio-signal to be measured.

Accordingly, by appropriately determining the target distance, the bio-signal detecting apparatus 20 may effectively differentiate the optical signal, which is emitted from the light sources positioned at distances other than the target distance and is detected; and in this manner, only the optical signal, having passed through the target depth, may be easily extracted, such that an accurate bio-signal may be detected. FIG. 2B illustrates a bio-signal detecting apparatus 21 according to another embodiment of the present disclosure.

Referring to FIG. 2B, each detection module included in the bio-signal detecting apparatus 21 includes a light source disposed at the center thereof, and one or more detectors disposed at equal intervals on a circumference centered on the light source. For example, the detection module of the bio-signal detecting apparatus 21 may have the arrangement structure in which the positions of the light source and the detector of each detection module included in the bio-signal detecting apparatus 20 may be exchanged with each other. Hereinafter, the repetitive arrangement structure of detection modules of the bio-signal detecting apparatus 21 is the same arrangement of the detector and the light source described above with reference to FIGS. 1B, 1C, and 2A, except that the light source is disposed at the position of the detector, and the detector is disposed at the position of the light source, such that overlapping description thereof will be omitted.

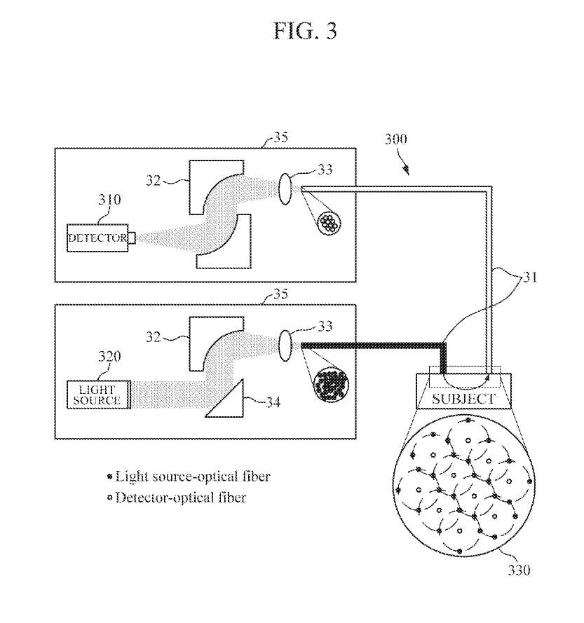

FIG. 3 is an exemplary diagram illustrating an example of a bio-signal detecting apparatus.

Referring to FIG. 3, a bio-signal detecting apparatus 300 includes a detector 310, a light source 320, and a detection module 330. The detector 310 and the light source 320 emit light on a subject through one or more optical fibers 31, and may detect light returning after being reflected or scattered from the subject. For example, the detection module 330 may emit light, emitted from the light source 320, on the subject through the optical fibers 31, and may transfer light, reflected and/or scattered from the subject, to the detector 310 through the optical fibers 31.

Further, the bio-signal detecting apparatus 300 includes a plurality of detectors and light sources, and one or more optical fibers, which interface with each detector and light source; and by individually controlling each detector and light source, the bio-signal detecting apparatus 300 may emit light on a subject and may detect light reflected or scattered from the subject.

In this case, the arrangement of the optical fibers 31 of the detection module 330, which interface with the subject at a distal end of the optical fibers 31, may be the same as the arrangement of the detector and light source of the detection module described above with reference to FIGS. 1B, 1C, 2A, and 2B, such that overlapping description thereof will be omitted.

The bio-signal detecting apparatus 300 may further include at least one of a parabolic mirror 21 and a lens 33, which focuses light, emitted from the light source 320, to a subject. For example, in the case where the bio-signal detecting apparatus 300 transfers light, emitted from the light source, through the optical fibers 31 and emits the light on the subject, the bio-signal detecting apparatus 300 may minimize light leakage by focusing the light to a distal end on one side of the optical fiber using at least one of the parabolic mirror 32 and the lens 33. Further, the bio-signal detecting apparatus 300 includes one or more mirrors 34 to change an optical path according to a structural arrangement of the light source 320.

In addition, the bio-signal detecting apparatus 300 may further include at least one of the parabolic mirror 32 and the lens 33 which focus the light reflected and/or scattered from a subject toward the detector 310. For example, when the bio-signal detecting apparatus 300 receives the light, reflected and/or scattered from the subject, through the optical fibers 31, and transfers the received light to the detector 310 which detects the light, light leakage may be minimized by focusing the light, received through the optical fibers, to the detector 310 using at least one of the parabolic mirror 32 and the lens 33. However, the bio-signal detecting apparatus 300 is not limited thereto, and may include one or more mirrors 34 to change an optical path according to a structural arrangement of the detector 310.

The detector 310 and the light source 320 of the bio-signal detecting apparatus 300 may be physically separated from each other by a light blocking part 35. For example, the detector 310 and/or the light source 320 of the bio-signal detecting apparatus 300 are physically separated from each other by being surrounded by a blocking module which blocks entry of an optical wavelength from the outside. In this manner, it is possible to fundamentally prevent unexpected noise from being included in the detected optical signal, the noise being caused when external light is introduced into the detector 310 or when light emitted from the light source 320 is scattered without passing through a subject and is introduced into the detector 310.

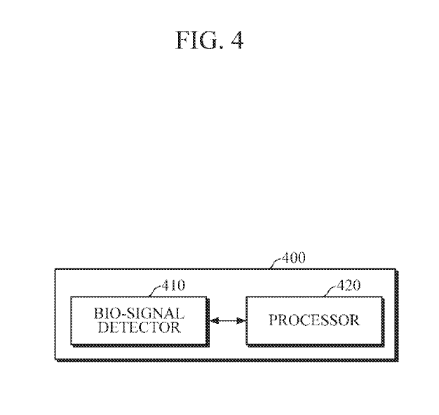

FIG. 4 is a block diagram illustrating an example of a bio-information processing apparatus.

Referring to FIG. 4, the bio-information processing apparatus 400 determines a target distance based on depth information of biomaterial to be measured, and detects light which is emitted from the light source disposed at the target distance and is reflected and/or scattered, to measure biomaterial positioned at the target depth.

Further, the bio-information processing apparatus 400 individually controls the detector and the plurality of light sources of each detection module, to generate a reference signal based on the light sources disposed at predetermined reference distances from the detector, and to correct the detected bio-signal.

The bio-information processing apparatus 400 may specify bio-information, including biomaterial information, by comparing the corrected bio-signal with bio-information correlation model.

The bio-information processing apparatus 400 may be implemented with a software module or manufactured in the form of a hardware chip to be embedded in various types of electronic apparatuses. Examples of the electronic apparatuses may include a cellular phone, a smartphone, a tablet PC, a laptop computer, a personal digital assistant (PDA), a portable multimedia player (PMP), a navigation, an MP3 player, a digital camera, a wearable device, and the like, and examples of the wearable device may include a watch-type device, wristband-type device, a ring-type device, a waist belt-type device, a necklace-type device, an ankle band-type device, a thigh band-type device, a forearm band-type device, and the like. However, the electronic device is not limited to these examples, and the wearable device is neither limited to the above examples.

Referring to FIG. 4, the bio-information processing apparatus 400 may include a bio-signal detector 410 and a processor 420. Here, the processor 420 may include one or more processors, a memory, and a combination thereof.

In the exemplary embodiment, the bio-signal detector 410 may measure a bio-signal by using a noninvasive method.

For example, the bio-signal detector 410 may measure a bio-signal by emitting light on a subject using an optical method, and detecting reflected light and/or scattered light. To this end, the bio-signal detector 410 may include a detection module.

In this case, the detection module of the bio-signal detector 410 includes the detector disposed at the center thereof; a plurality of light sources disposed on a circumference centered on the detector; other detection modules, adjacent to any one detection module, are arranged so that circumferences thereof intersect each other; and light sources are positioned at each intersection point. For example, the bio-signal detector 410 may have the same structure as that of the bio-signal detecting apparatus described above with reference to FIGS. 1A, 1B, 1C, 2A, and 2B, such that overlapping description thereof will be omitted.

The processor 420 controls the bio-signal detector 410 to activate only the light sources positioned at the plurality of reference distances from any one detection module. Here, the reference distance refers to a distance to a light source from any one detector included in the bio-signal detector 410. In the case where the bio-signal detector 410 includes a plurality of detection modules, the reference distance may be a distance to each light source from any one detector of the plurality of detection modules, in which case one or more reference distances may be calculated.

For example, the processor 420 may detect an optical signal by activating only one detector of the bio-signal detector 410, and by activating only the light sources positioned at equal distances from the activated detector. In this case, the processor 420 may generate a reference signal for the reference distance by using the detected optical signal.

Further, in the case where there are a plurality of detection modules, and one or more reference distances are calculated, the processor 420 may sequentially activate the light sources positioned at equal distances from any one detector, and may generate the reference signal for each reference distance based on the detected optical signal.

Based on the generated reference signal, the processor 420 may correct a bio-signal obtained from the bio-signal detector 410.

For example, the optical signal detected by the bio-signal detector 410 may be an optical signal detected by the detector while all the detectors and the light sources are activated. Here, the detected optical signal may be an optical signal of light emitted from the light sources positioned at a plurality of distances from each detector; and the processor 420 may analyze the optical signal, detected by the bio-signal detector 410, based on the generated reference signal, and may classify the detected optical signals by reference distances.

For example, among optical signals classified by distances, in response to determination that an optical signal is an optical wavelength emitted at the reference distance other than the target distance, the processor 420 may correct the bio-signal obtained from the bio-signal detector 410 by removing the signal and thus extracting only the signal at the target distance.

However, the processor 420 is not limited thereto, and generation of the reference signal and correction of the bio-signal by the processor 420 may be selectively performed. For example, the processor 420 may omit the processes of reference signal generation and bio-signal correction; and the processor 420 may generate a reference signal during the initialization of the bio-information processing apparatus, and may omit the generation of the reference signal during bio-information processing thereafter. Further, the processor 420 may receive a reference signal pre-generated for each reference distance from an external device, and may use the received reference signal to correct and process the bio-signal.

As described above, by omitting the processes of reference signal generation and bio-signal correction, the processor 420 may output quick results in response to a user's bio-signal processing command; and the processor 420 may generate a reference signal for each distance for a user of the bio-signal processing apparatus 400, and may correct the obtained bio-signal, to measure more personalized bio-information.

The processor 420 may measure bio-information based on the bio-signal corrected by using a bio-information correlation module.

In the exemplary embodiment, by using the bio-information correlation module generated based on the correlation between a biomaterial change and an optical wavelength change, the processor 420 may measure bio-information of a user.

Here, the biomaterial is skin material including moisture, protein, lipid, or various minerals, and may include at least one of blood glucose, cholesterol, and neutral fats as blood components.

Further, the bio-information correlation model may be a correlation model pre-generated based on a correlation between a biomaterial change and a spectrum data change by using spectrum data of the detected optical signal. The bio-information correlation model may be classified by user information, including age, gender, weight, Body Mass Index (BMI), and health information, and by bio-signal measurement points.

For example, the bio-information correlation model may be a correlation model between a blood glucose change and near-infrared (NIR) spectrum data change, and may be a correlation model pre-generated based on spectrum data measured on an outer side of a wrist of a man in his thirties. However, the bio-information correlation model is not limited thereto, and the processor 420 may measure bio-information by selecting a bio-information correlation model suitable for the types of bio-information to be measured, and by comparing the selected bio-information correlation model with the detected bio-signal.

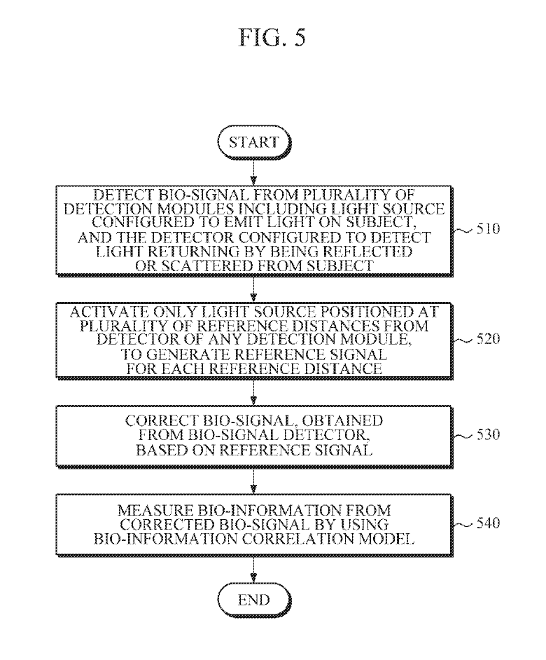

FIG. 5 is a flowchart illustrating an example of a bio-information processing method.

The bio-information processing method of FIG. 5 may be performed by the bio-information processing apparatus 400 illustrated in FIG. 4.

The bio-information processing apparatus 400 may detect a bio-signal from the plurality of detection modules including a light source which emits light on a subject, and the detector which detects light that returns by being reflected or scattered from a subject in operation 510.

For example, the bio-information processing apparatus 400 includes the detector disposed at the center thereof, and a plurality of light sources disposed on a circumference centered on the detector; other detection modules, adjacent to any one detection module, are arranged so that circumferences thereof intersect each other; and the light sources are positioned at each intersection point. In this case, the bio-information processing apparatus 400 may detect a bio-signal by detecting light, which is emitted from the light sources and returns after being reflected and/or scattered from the subject.

When detecting a bio-signal, the bio-information processing apparatus 400 may activate only the light sources positioned at the plurality of reference distances from a detector of any detection module, to generate a reference signal for each of the reference distances in operation 520.

For example, in the case where there is one or more reference distances, the bio-information processing apparatus 400 sequentially activate the light sources positioned at equal distances from any one detector, and may generate the reference signal for each reference distance based on the detected optical signal.

The bio-information processing apparatus 400 may correct the bio-signal, obtained from the bio-signal detector, based on the generated reference signal in operation 530.

For example, the optical signal detected by the bio-information processing apparatus 400 may be an optical signal detected by the detector while all the detectors and the light sources are activated. Here, the detected optical signal may be an optical signal emitted from the light sources positioned at a plurality of distances from each detector; and the bio-information processing apparatus 400 may analyze the detected optical signal based on the generated reference signal, and may classify the detected optical signals by reference distances.

Upon classifying the detected optical signals by reference distances, the bio-information processing apparatus 400 may correct the obtained bio-signal by removing the optical signal, which is determined to be an optical wavelength emitted at the reference distance other than the target distance, and thus by extracting only the signal at the target distance.

The bio-information processing apparatus 400 may measure bio-information from the corrected bio-signal by using the bio-information correlation model in operation 540.

For example, by using the bio-information correlation model generated based on the correlation between a biomaterial change and an optical wavelength change, the bio-information processing apparatus 400 may measure bio-information of a user.

While not restricted thereto, an exemplary embodiment can be embodied as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, an exemplary embodiment may be written as a computer program transmitted over a computer-readable transmission medium, such as a carrier wave, and received and implemented in general-use or special-purpose digital computers that execute the programs. Moreover, it is understood that in exemplary embodiments, one or more units of the above-described apparatuses and devices can include circuitry, a processor, a microprocessor, etc., and may execute a computer program stored in a computer-readable medium.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. A bio-signal detecting apparatus comprising:
a plurality of detection modules, each detection module of the plurality of detection modules comprising a detector disposed at a center thereof and a plurality of light sources disposed at a circumference centered at the detector,
wherein the plurality of detection modules comprise a first detection module and a second detection module that is disposed adjacent to the first direction module, a circumference of the first detection module intersects with a circumference of the second detection module, and one or more light sources of the plurality of light sources are disposed at intersection points where the circumference of the first detection module intersects with the circumference of the second detection module.

2. The apparatus of claim 1, wherein the first detection module and the second detection module share the one or more of the plurality of light sources disposed at the intersection points where the circumference of the first detection module with the second detection module;
the plurality of detection modules further comprise a third detection module that is disposed adjacent to the first detection module; and
a light source of the first detection module and a light source of the third detection module are disposed respectively at two intersection points where the circumference of the first detection module intersects with a circumference of the third detection module.

3. The apparatus of claim 2, wherein the one or more light sources of the plurality of light sources shared by the first detection module and the second detection module are disposed at the circumference of the third detection module.

4. The apparatus of claim 1, wherein a target distance between the detector and the plurality of light sources of each of the plurality of detection modules is set based on a target depth from a surface of a subject to a location where a target biomaterial of the subject is present.

5. The apparatus of claim 4, wherein the target distance is approximately 0.65 mm.

6. The apparatus of claim 1, wherein a number of the plurality of light sources of each detection module is four and the plurality of light sources are disposed at an equal interval on the circumference of each of the plurality of detection modules.

7. The apparatus of claim 1, wherein the plurality of light sources and the detector are physically separated by a light blocking part.

8. The apparatus of claim 7, further comprising at least one of a parabolic mirror and a lens which focuses a light emitted from the plurality of light sources on a subject.

9. The apparatus of claim 7, further comprising at least one of a parabolic mirror and a lens which focuses a light returning from a subject toward the detector.

10. A bio-signal detecting apparatus comprising:
a plurality of detection modules, each detection module of the plurality of detection modules comprising a light source disposed at a center thereof, and a plurality of detectors disposed at a circumference centered at the light source,
wherein the plurality of detection modules comprise a first detection module and a second detection module that is disposed adjacent to the first detection module, a circumference of the first detection module intersects with a circumference of the second detection module, and one or more detectors of the plurality of detectors are disposed at intersection points where the circumference of the first detection module intersects with the circumference of the second detection module.

11. The apparatus of claim 10, wherein the first detection module and the second detection module share the one or more detectors of the plurality of detectors disposed at the intersection points where the circumference of the first detection module with the second detection module;
the plurality of detection modules further comprise a third detection module that is disposed adjacent to the first detection module; and
a detector of the first detection module and a detector of the third detection module are disposed respectively at two intersection points where the circumference of the first detection module intersects with a circumference of the third detection module.

12. The apparatus of claim 11, wherein the one or more detectors of the plurality of detectors shared by the first detection module and the second detection module are disposed at the circumference of the third detection module

13. A bio-information processing apparatus comprising:
a bio-signal detector that comprises a plurality of detection modules, each of the plurality of detection modules comprising a plurality of light sources configured to emit light onto a subject, and a detector configured to detect light returning from the subject; and
a processor configured to analyze a bio-signal obtained from the bio-signal detector and process bio-information,
wherein the detector is disposed at a center of a corresponding one of the plurality of detection modules, and the plurality of light sources are disposed at a circumference centered on the detector, and the plurality of detection modules comprise a first detection module and a second detection module that is disposed adjacent to the first detection module, wherein a circumference of the first detection module intersects with a circumference of the second detection module, and one or more light sources of the plurality of light sources are disposed at intersection points where the circumference of the first detection module intersects with the circumference of the second detection module.

14. The apparatus of claim 13, wherein the processor is further configured to control the bio-signal detector and activate only light sources positioned at a plurality of reference distances from the detector, among the plurality of the light sources, to generate a reference signal for each of the plurality of reference distances.

15. The apparatus of claim 13, wherein the processor is further configured to correct the bio-signal based on the generated reference signal.

16. The apparatus of claim 15, wherein the processor is further configured to obtain the bio-information based on the corrected bio-signal by using a bio-information correlation model.

17. The apparatus of claim 13, wherein the bio-information comprises information of at least one of moisture, protein, lipid, minerals, blood glucose, cholesterol, and neutral fats.

18. A bio-information processing method comprising:
detecting a bio-signal by a plurality of detection modules, each of the plurality of module including a plurality of light sources configured to emit light on a subject, and a detector configured to detect the light returning from the subject; and
analyzing the detected bio-signal to process bio-information,
wherein the detector is disposed at a center of a corresponding one of the plurality of detection modules, and the plurality of light sources are disposed at a circumference centered on the detector, and the plurality of detection modules comprise a first detection module and a second detection module that is disposed adjacent to the first detection module, and
wherein a circumference of the first detection module intersects with a circumference of the second detection module, and one or more light sources of the plurality of light sources are disposed at intersection points where the circumference of the first detection module intersects with the circumference of the second detection module.

19. The method of claim 18, wherein the analyzing the bio-information comprises:
activating only light sources positioned at a plurality of reference distances from the detector, among the plurality of the light sources, and
generating a reference signal for each of the plurality of reference distances.

20. The method of claim 18, wherein the analyzing the bio-information comprises correcting the bio-signal based on the generated reference signal.

21. The method of claim 18, wherein the analyzing the bio-information comprises obtaining the bio-information based on the corrected bio-signal by using a bio-information correlation model.
